(19) United States
(12) Patent Application Publication
(10) Pub. No.: US 2022/0257511 A1
(43) Pub. Date: Aug. 18, 2022

(54) NANOVESICLES

(71) Applicant: Loughborough University, Loughborough Leicestershire (GB)

(72) Inventors: Danish MALIK, Loughborough Leicestershire (GB); Salvatore CINQUERRUI, Loughborough Leicestershire (GB)

(21) Appl. No.: 17/622,384

(22) PCT Filed: Jun. 26, 2020
(86) PCT No.: PCT/GB2020/051555

§ 371 Date: Dec. 23, 2021

(30) Foreign Application Priority Data
Jun. 27, 2019 (GB) 1909242.8

(51) Int. Cl. A61K 9/127; A61K 35/76; A61K 38/47

(52) U.S. Cl. CPC A61K 9/127; A61K 48/00; A61K 38/47; A61K 35/76

(57) ABSTRACT

A process for the preparation of unilamellar vesicles, wherein a unilamellar vesicle comprises an amphiphilic membrane enclosing an aqueous core; and compositions comprising unilamellar vesicles.

The process comprises providing a primary emulsion comprising an amphiphilic membrane forming component, a first aqueous phase W1 and a first oil phase O1. The first aqueous phase W1 is dispersed as droplets in the oil phase O1 such that the primary emulsion is a water-in-oil emulsion. The primary emulsion comprises droplets having a mean diameter of less than 1000 nm.

The process comprises combining the primary emulsion with a second aqueous phase W2 to produce a secondary emulsion that is a water-in-oil-in-water emulsion; dewetting to yield unilamellar vesicles in the secondary emulsion; and isolating the unilamellar vesicles.

### FIELD OF THE INVENTION

The invention relates to nanovesicles, their preparation and use.

### BACKGROUND

A vesicle is a closed structure formed by an amphiphilic membrane enclosing a solvent core (usually water). Vesicles can be prepared artificially and also form naturally during the processes of secretion (exocytosis), uptake (endocytosis) and transport of materials within the plasma membrane.

The structure of the amphiphilic membrane can be a bilayer or a single monolayer, depending on the type of membrane-forming amphiphile used for vesicle generation. Membranes made from phospholipids or di-block copolymers have a bilayer structure, which resembles the structure of cell membranes, whereas membranes made from tri-block copolymers may have a single monolayer structure (Huang et al.).

Processes have been developed for producing liposomes for encapsulation of therapeutic agents, including thin film hydration methods, reverse phase evaporation, detergent depletion, ethanol injection, emulsification, supercritical phase formation, membrane extrusion, and rapid solvent exchange. However, known methods suffer from low encapsulation efficiency, particularly for the encapsulation of large nanometre sized entities. The aforementioned methods also tend to produce multilamellar vesicles with little control over the chemical composition of adjacent bilayers (Szoka and Papahadjopoulos, 1978).

As far as the inventors are aware, there are no reliable methods to produce single unilamellar vesicles in the sub-micron size range that can encapsulate large nanoparticles in a highly controlled manner and with high encapsulation efficiency e.g. bacteriophages (˜50-200 nm in size), plasmids (˜100 nm), CRISPR guide RNAs (˜10-20 nm) etc.

### SUMMARY OF THE INVENTION

According to a first aspect of the invention there is provided a process for the preparation of unilamellar vesicles, a unilamellar vesicle comprising an amphiphilic membrane enclosing an aqueous core; the process comprising

providing a primary emulsion comprising an amphiphilic membrane forming component, a first aqueous phase W1 and a first oil phase O1; the first aqueous phase W1 being dispersed as droplets in the first oil phase O1 such that the primary emulsion is a water-in-oil emulsion and wherein the primary emulsion comprises droplets having a mean diameter of less than 1000 nm;
combining the primary emulsion with a second aqueous phase W2 to produce a secondary emulsion that is a water-in-oil-in-water emulsion;
dewetting to yield unilamellar vesicles in the secondary emulsion; and isolating the unilamellar vesicles.

The inventors have determined that the process of the invention provides controlled de-wetting of a secondary emulsion to yield unilamellar vesicles: a unilamellar vesicle comprises a single amphiphilic membrane enclosing an aqueous core. Dewetting may be viewed as controlled dewetting or allowing de-wetting to take place.

Using this knowledge, the inventors have determined that the vesicles can be isolated from the secondary emulsion. For example, differences in density can be utilised, including differential centrifugation, such as employing equilibrium density gradients.

According to a specific embodiment of the first aspect of the invention there is provided a process for the preparation of unilamellar vesicles, the process comprising providing a primary emulsion comprising an amphiphilic membrane forming component, a first aqueous phase W1 and a first oil phase O1; the first aqueous phase W1 being dispersed as droplets in the first oil phase O1 such that the primary emulsion is a water-in-oil emulsion and wherein the primary emulsion comprises droplets having a mean diameter of less than 1000 nm;

combining the primary emulsion with a second aqueous phase W2 to produce a secondary emulsion that is a water-in-oil-in-water emulsion;

dewetting to yield unilamellar vesicles in the secondary emulsion; and separating the secondary emulsion into layers of different densities, at least one layer comprising the first oil phase; at least one layer comprising the second aqueous phase; and at least one layer comprising unilamellar vesicles, each unilamellar vesicle comprising an amphiphilic membrane enclosing an aqueous core.

The inventors have determined that the process of the invention provides controlled de-wetting of a secondary emulsion to yield unilamellar vesicles: a unilamellar vesicle comprises a single amphiphilic membrane enclosing an aqueous core. Using this knowledge, the inventors have determined that the vesicles can be separated using density, including differential centrifugation such as employing equilibrium density gradients. Dewetting may be viewed as controlled dewetting or allowing de-wetting to take place.

It will be understood that the first aqueous phase W1 forms the aqueous core. The unilamellar vesicles can be further processed as described below to generate multilamellar vesicles (including bilamellar vesicles).

According to a second aspect of the invention there is provided a composition comprising layers of different densities;

at least one layer comprising an oil phase (O1);

at least one layer comprising an aqueous phase (W2); and

at least one layer comprising a plurality of unilamellar vesicles, the unilamellar vesicles each comprising an amphiphilic membrane enclosing an aqueous core and having a mean diameter of less than 1000 nm.

The layers may be arranged in a container (e.g. a centrifuge tube) with the densest layer at the bottom and the least dense layer at the top.

It will be understood that the composition of the second aspect is producible by the process of the first aspect. Moreover, at least one unilamellar vesicle may encapsulate a nanoparticle.

The composition may comprise two layers that comprise a plurality of unilamellar vesicles. One layer may comprise unilamellar vesicles that encapsulate a nanoparticle, i.e. that have a cargo. Another layer may comprise unilamellar vesicles that do not encapsulate a nanoparticle, i.e. without a cargo. Since the presence of the nanoparticle affects the density of the vesicle, this provides a convenient way to separate the vesicles carrying a cargo from “empty” vesicles.

According to a third aspect of the invention there is provided a unilamellar vesicle comprising an amphiphilic membrane enclosing an aqueous core, the unilamellar vesicle having a diameter of less than 1000 nm and encapsulating a nanoparticle.

It will be understood that the second aspect of the invention may comprise a plurality of vesicles according to the third aspect of the invention.

According to a fourth aspect of the invention there is provided a multilamellar vesicle comprising two or more amphiphilic membranes successively enclosing an aqueous core, the multilamellar vesicle having a diameter of less than 1000 nm and encapsulating a nanoparticle having a mean diameter of 10 to 200 nm.

It will be understood that the two or more amphiphilic membranes are concentric around an aqueous core.

According to a fifth aspect of the invention there is provided a multivesicular vesicle comprising an outer amphiphilic membrane enclosing two or more unilamellar vesicles, the unilamellar vesicles having a diameter of less than 1000 nm and encapsulating a nanoparticle having a mean diameter of 10 to 200 nm.

It will be appreciated that the invention also resides in the compositions of the invention for use as a medicament.

The controlled production of multivesicular vesicles allows different compositions of the smaller internal vesicles to be incorporated into the bigger (outer) vesicle. This is relevant for gene therapy. The smaller inner vesicles can diffuse faster than a large vesicle once inside a cell. Additionally, the inner small vesicles may be decorated with features that improve targeting or allow removal if not encapsulated to ensure delivery only in certain cells etc.

The process of the invention allows nanoencapsulation of shear sensitive therapeutic agents including large nanoparticles, such as bacteriophages, plasmids, gene vectors such as DNA and RNA, CRISPR RNA guides, vaccines etc. It will be understood that the first aqueous phase (WI) droplets serve as templates for the resulting vesicles. Vesicles having a diameter of less than 1000 nm can be described as nanoscale vesicles, submicron vesicles or nanovesicles.

Huang et al. is a review summarizing the available technologies employing emulsion drops as vesicle templates. Huang states that conventional methods for vesicle production, such as hydration, provide very little control over the process since they are based on spontaneous assembly of amphiphiles in an aqueous environment. Huang discusses use of microfluidic devices to form unilamellar vesicles produced through formation of double emulsions and the subsequent removal of the organic solvent e.g. by evaporation. The vesicles formed in microfluidic devices are at the micrometre length scale and are not nanovesicles.

Deng et al. describes the preparation of single bilayer liposomes having diameters from 25 to 190 μm (micron), which are much larger than the vesicles of the present invention. By changing the dimensions of the templates, Deng teaches that liposomes ranging from 20 to 200 μm can be easily formed in capillary microfluidic devices. There is no suggestion that smaller liposomes can be obtained. The liposomes are prepared by dewetting W/O/W double emulsions by means of a triblock copolymer surfactant (Pluronic F-68) to adjust interfacial energies. In the absence of the surfactant the emulsion is stable and no dewetting is reported.

Ding et al. describes the preparation of W1/O/W2 double nanoemulsions by means of an original two-step approach. A primary water-in-oil emulsion is prepared by high-pressure homogenization, followed by re-emulsification of the primary emulsion by a low energy method to preserve the double emulsion nanostructure. The aqueous phase is composed of Milli-Q water, maltodextrin (thickener), carboxyfluorescein (probe), and potentially acrylamide (monomer), crosslinker (MBA), and photoinitiator (DMHA). The oil phase is composed of Labrafac WLs (medium chain triglycerides) and PGPR (low-HBL stabilizer). The size distribution of the primary and secondary emulsion remained unchanged after several weeks of storage. Hence there is no suggestion that the nanoemulsion undergoes dewetting.

US2012/0225117 describes a process for producing liposomes comprising a step of removing an organic solvent contained in a W1/O/W2 emulsion. The inventors submit that the removal of the solvent will hinder the controlled production of unilamellar vesicles, with remaining lipids in the oil phase forming multivesicular liposomes in an uncontrolled manner. To minimise the formation of multilamellar and/or multivesicular liposomes the amount of lipids is minimised. The inventors submit that there is just enough to make the unilamellar vesicles, which will affect the stability of the formed liposomes. This could affect the encapsulation yield or leakage or bursting. In addition, US2012/0225117 process employs a secondary membrane to form the W1/O/W2 emulsion and this is not required, so it is unduly complex.

Low shear mixing of a primary W1/O nanoemulsion with an excess of secondary aqueous phase W2 results in formation of unilamellar sub-micron vesicles with high encapsulation efficiency of agent dissolved or dispersed in W1 phase. This is an unexpected result.

The inventors have determined that there is no need to create a thin nanometre length scale oil film in the double emulsion for dewetting to occur. Surprisingly, the double emulsion shell thickness is not important for the formation of unilamellar vesicles.

Moreover, the inventors have determined that there is no need to have just one water droplet in each double emulsion droplet. Surprisingly, at the nanometre length-scale the inventors have determined that multiple interior W1 nanodroplets in a larger micron-sized double emulsion (referred to as multiple emulsions) drop still undergoes dewetting to give unilamellar nanometer sized liposomes.

Significantly, the inventors have determined that there is no requirement for evaporation of the oil phase to generate the vesicles.

Dewetting and Isolation

The secondary emulsion is detwetted to yield unilamellar vesicles in the secondary emulsion. Generally, dewetting describes the process of retraction of a fluid from a non-wettable surface it was forced to cover. In the present invention, the inventors propose that a portion of the oil phase, e.g. a water-soluble organic solvent, migrates out of the oil phase and this depletion leads the remaining oil phase components to separate, perhaps through a difference in density or due to Brownian thermal motion.

In US2012/0225117, de-wetting is achieved by evaporation of the organic solvent from the W1/O/W2 emulsion. The present invention does not require removal of the organic solvent from the emulsion/evaporation; the unilamellar vesicles are generated within the secondary emulsion.

Dewetting the secondary emulsion may comprise the use of an agent to trigger dewetting. The agent may be present in the second aqueous phase. The agent may be a polymer surfactant (e.g. a poloxmer), as discussed in more detail below.

Isolation of the Vesicles

Isolating the unilamellar vesicles may comprise filtration, such as filtration through nanopores. For example, the process may comprise filtration of the second emulsion through nanopores whilst retaining vesicles (e.g. liposomes) and washing using diafiltration.

Isolating the unilamellar vesicles may comprise chromatography, such as size exclusion chromatography, liquid chromatography or field-flow fractionation.

Isolating the unilamellar vesicles may comprise the use of a salt gradient in a capillary channel. For example, the vesicles may be trapped in nanochannels based on their size and surface charge (Rasmussen M K, et al.).

Isolating the unilamellar vesicles may comprise separating the secondary emulsion into layers of different densities, at least one layer comprising the first oil phase; at least one layer comprising the second aqueous phase; and at least one layer comprising unilamellar vesicles, each unilamellar vesicle comprising an amphiphilic membrane enclosing an aqueous core.

In one embodiment, the secondary emulsion containing the vesicles is separated into layers having different densities. The differences in the densities of the components of the secondary emulsion (oil, water and vesicles) can be exploited to separate out the unilamellar vesicles without the need for solvent evaporation. Over time, the most dense components will settle to the bottom of a container (e.g. a centrifuge tube) and the least dense components will rise to the top. This process can be accelerated by use of a centrifuge.

A centrifuge is an apparatus consisting essentially of a compartment (e.g. a centrifuge tube) spun about a central axis. Relative centrifugal force (RCF) describes the amount of force of acceleration applied to a sample in a centrifuge.

The secondary emulsion may be centrifuged (subjected to the action of a centrifuge) to produce the layers having different densities. The process may comprise a step of transferring a portion of the secondary emulsion to a centrifuge tube (e.g. a 50 ml centrifuge tube). The secondary emulsion may be centrifuged with a density gradient or without a density gradient.

An (equilibrium) density gradient can be used. An equilibrium density gradient is made by layering solutions (e.g. aqueous sucrose or CsCl) of decreasing concentrations into a centrifuge tube to create a gradient of decreasing density. The secondary emulsion (or a portion thereof) may be carefully layered on the top of the density gradient. Typically 10 ml of sample is layered on a 40 ml gradient.

The resulting sample (comprising the secondary emulsion and the density gradient) may be allowed to settle at room temperature for a given period.

Alternatively, the resulting sample may be centrifuged (e.g. at 50,000 g) for at least 1 hour, at least 2 hours or at least 3 hours and/or for no more than 72 hours, 24 hours or 12 hours.

The sample (comprising the secondary emulsion and the density gradient) may be centrifuged at a RCF (relative centrifugal force) of at least at least 50,000 g, at least 200,000 g or at least 600,000 g and/or the sample may be centrifuged at a RCF of 600,000 g or less, 300,000 g or less or 100,000 g or less.

A vesicle rich layer occurs as a band in the density gradient and can be isolated, e.g. by removing/decanting a supernatant layer (e.g. the oil layer) or by syringe. The isolated vesicle rich layer can then be re-suspended in a buffer, i.e. a buffer solution such as Tris-HCl buffer. A buffer solution (a pH buffer or hydrogen ion buffer) is an aqueous solution consisting of a mixture of a weak acid and its conjugate base, or vice versa. Its pH changes very little when a small amount of strong acid or base is added to it. A dialysis step may be used to purify the vesicle sample.

The secondary emulsion (or a portion thereof) may be centrifuged without a density gradient to generate layers of different densities. If desired, an additional component having a known density (e.g. a 100 mM sucrose solution) can be added after centrifugation to help separate the layers.

Substances to be Encapsulated

The process of the present invention can be employed to easily encapsulate substances by dissolving or dispersing the substance in the first aqueous phase prior to formation of the primary emulsion.

The substances to be encapsulated are not specifically restricted and include substances known in the fields of medicines, cosmetics, food etc. The invention is particularly useful for encapsulating nanoparticles, especially large nanoscale nanomedicine substances, such as bacteriophages, plasmids, and gene vectors. Encapsulation in vesicles may allow targeted delivery of these nanomedicines e.g. to treat intracellular bacterial infections e.g. to treat tuberculosis or listeriosis or to deliver DNA to the nucleus for gene therapy.

The first aqueous phase may comprise an active pharmaceutical ingredient, such as a water-soluble drug. In this way the vesicles can be employed for medical treatment.

Suitable active ingredients include anti-tumour agents, antibacterial agents, vitamins, nucleic acid (sense strand or antisense strand of DNA or RNA, plasmid, vector, mRNA, siRNA etc.), proteins (enzymes including hemolysins, antibody, peptide, Cas proteins such as Cas9, zinc finger nucleases, etc), bacteriophages including engineered bacteriophages (e.g. encoding CRISPR/Cas system encoding Cas endonucleases), and vaccine formulations. Active ingredients could be encapsulated individually or in combination, i.e. co-encapsulation.

The first aqueous phase may comprise a bacteriophage such that the resulting vesicle encapsulates a bacteriophage. Examples of bacteriophage families are listed below.

| Family name | Morphology | Genome | Examples |
|---|---|---|---|
| Corticoviridae | Icosahedral capsid with lipid | dsDNA | |
| | layer | | |
| Cystoviridae | Enveloped, icosahedral capsid, | dsRNA | |
| | lipids | | |
| Fuselloviridae | Pleomorphic, envelope, lipids, | dsDNA | |
| | no capsid | | |
| Inoviridae | Rod-shaped with helical | ssDNA | M13 |
| | symmetry | | |
| Leviviridae | Quasi-icosahedral capsid | ssRNA | MS2 |
| Lipothrixviridae | Enveloped filaments, lipids | dsDNA | |
| Microviridae | Icosahedral capsid | ssDNA | Phi X 174 |
| Myoviridae | Contractile tail | dsDNA | T4 |
| Plasmaviridae | Pleomorphic, envelope, lipids, | dsDNA | |
| | no capsid | | |
| Podoviridae | Short, non-contractile tail | dsDNA | T7, T3 |
| Rudiviridae | Helical rods | dsDNA | |
| Siphoviridae | Long, non-contractile tail | dsDNA | Lambda, |
| | | | D29, T5 |
| Tectiviridae | Icosahedral capsid with inner | dsDNA | |
| | lipoprotein vesicle | | |

ds = double stranded,
ss = single stranded

The aqueous phase may comprise a bacteriophage, which is ˜50-200 nm in size.

The aqueous phase may comprise a Myoviridae bacteriophage, such as Myovirus Staphylococcus aureus phage K.

The aqueous phase may comprise an endolysin. Endolysins, also known as phage lysins or murein hydrolases, are hydrolytic enzymes produced by bacteriophages in order to cleave the host's cell wall during the final stage of the lytic cycle. Examples of endolysins are provided below.

| Bacteria | Phage | Endolysin |
|---|---|---|
| Streptococcus pneumoniae | Cp-1 | Cp1-1 |
| Streptococcus pneumonia | Dp-1 | PAL |
| Streptococcus pyogenes | C1 | C1 |
| Streptococcus agalactiae | NCTC 11361 | PlyGBS |
| Bacillus anthracis | γ | PlyG |
| | | PlyPH |
| Staphylococcus aureus | MR11 | MV-L |
| | | ClyS |
| | Bacteriophage K | CHAPk |
| | GH15 | LysGH15 |

Table adapted from Daniel C. Nelson, et al., in Advances in Virus Research, 2012

The first aqueous phase may comprise a protein, such as an enzyme, such that the resulting vesicle encapsulates the protein e.g. Cas9 endonuclease (size ˜20 nm).

The first aqueous phase may comprise a haemolysin, such as LLO. Listeriolysin O (LLO) is produced by the bacterium Listeria monocytogenes, the pathogen responsible for causing listeriosis. The LLO can be employed to break the amphiphilic membrane and release the contents encapsulated within the vesicle. The first aqueous phase may comprise a haemolysin, such as LLO, in addition to another component.

The resulting unilamellar vesicle may co-encapsulate a hemolysin and another component, such as an endolysin.

The first aqueous phase may comprise a gene vector, such as DNA or RNA, such that the resulting vesicle encapsulates the gene vector.

The first aqueous phase may comprise a plasmid, i.e. a small DNA molecule within a cell that is physically separated from chromosomal DNA and can replicate independently. The plasmid may have a size of ˜100 nm.

Vesicles

A vesicle comprises an amphiphilic membrane enclosing an aqueous core.

The process of the present invention yields unilamellar vesicles, i.e. where the amphiphilic membrane consists of a single monolayer or a single bilayer. The sizes of small unilamellar vesicles (SUVs) range from 10 to 100 nm, whereas the sizes of large unilamellar vesicles (LUVs) range from 100 nm to 1 μm.

The unilamellar vesicles of the present invention have a mean diameter of less than 1000 nm and can be described as nanoscale vesicles, submicron vesicles or nanovesicles.

The unilamellar vesicles may have a mean diameter of 900 nm or less, 800 nm or less, 700 nm or less, 600 nm or less, 500 nm or less, 400 nm or less, 300 nm or less or 200 nm or less; and/or the unilamellar vesicles may have a mean diameter of 10 nm or more, 50 nm or more, 100 nm or more, 200 nm or more, 300 nm or more, 400 nm or more, 500 nm or more, 600 nm or more or 700 nm or more. The unilamellar vesicles may have a mean diameter of from 100 to 600 nm.

It will be understood that “mean diameter” refers to a volume mean particle diameter. The volume mean diameter value of the vesicles can be measured using Nanoparticle Tracking size analysis (NTA LM10 Malvern Pananalytical, UK) based on direct observation and measurement of particle diffusion.

The unilamellar vesicles can be further processed to generate multilamellar vesicles, i.e. where the amphiphilic membrane consists of two or more successive monolayers/bilayers. The process of the first aspect is repeated with the unilamellar vesicles as part of a third aqueous phase, which is emulsified with a second oil phase and then a fourth aqueous phase to generate another water-in-oil-in-water emulsion.

A multilamellar vesicle (e.g. liposome or polymersome) can be viewed as a unilamellar vesicle having at least one further amphiphilic membrane surrounding the aqueous core. The amphiphilic membranes successively surround the aqueous core, i.e. they are concentric around the core.

The regions between the successive amphiphilic membranes may be described as intra-lamellar compartments or shells. If there is just one additional amphiphilic membrane (i.e. two in total) then the multilamellar vesicle may be described as a bilamellar vesicle having an inner amphiphilic membrane surrounding a core and an outer amphiphilic membrane surrounding the shell. The aqueous core can be different from the shell.

A multilamellar vesicle can be distinguished from a multivesicular vesicle. A multivesicular vesicle can viewed as two or more unilamellar vesicles that are enclosed by an outer amphiphilic membrane.

Further Processing

Unilamellar vesicles produced according to the first aspect of the invention may be further processed to yield multilamellar (including bilamellar) vesicles. Multilamellar vesicles with tailored chemistries for each shell may allow targeting of different cell compartments e.g. the outer shell may allow fusion with the outer cell membrane to traffic the vesicle within the cytosol whereas the inner shell may deliver the therapeutic cargo by fusing with the cell nucleus suitable for gene therapy.

The unilamellar vesicles may be suspended in a third aqueous phase, which may contain a buffer such as Tris-HCl. The third aqueous phase may be combined with a second oil phase to generate a water-in-oil emulsion. This water-in-oil emulsion can then be combined with a fourth aqueous phase to form a further double emulsion and thereby yield bilamellar vesicles (having two amphiphilic membranes).

The bilamellar vesicles may be suspended in a fifth aqueous phase, which may contain a buffer such as Tris-HCl. The fifth aqueous phase may be combined with a third oil phase to generate a water-in-oil emulsion. This water-in-oil emulsion can then be combined with a sixth aqueous phase to form a further double emulsion and thereby yield multilamellar vesicles (having three successive amphiphilic membranes).

Primary Emulsion

The primary emulsion comprises the amphiphilic membrane forming component, the first aqueous phase W1 and the oil phase O.

The primary emulsion can be described with reference to the amount of amphiphilic membrane forming component contained therein. The primary emulsion may comprise at least 1 wt %, at least 2 wt % at least 3 wt %, at least 5 wt % or at least 10 wt % amphiphilic membrane forming component; and/or the primary emulsion may comprise 20 wt % or less, 10 wt % or less, 8 wt % or less or 5 wt % or less amphiphilic membrane forming component. The primary emulsion may comprise from 1 to 8 wt % amphiphilic membrane forming component.

The desired size of the vesicle may determine the amount of amphiphilic component that will allow formation of the vesicle e.g. for a 100 nm liposome, a minimum of 0.142 mmoles may be required whereas for a 500 nm liposome, a minimum of 0.028 mmoles (of DPPC etc.) may be required. These values were calculated based on stearic considerations, spherical configuration of the bilayer lipid membrane and carrying out a lipid mass balance.

The first aqueous phase W1 is dispersed as nanometre length scale droplets in the oil phase such that the primary emulsion can be described as a water-in-oil nanoemulsion.

The primary emulsion can be described with reference to the volume ratio of the first aqueous phase W1 to the oil phase O1. The volume ratio W1:O1 may be 1 to 40% W1:99 to 60% O1; 2 to 30% W1:98 to 70% O1; 3 to 20% W1:97 to 80% O1; or 5 to 10% W1:95 to 90% O1. The volume ratio W1:O1 may be 1 to 10% W1:99 to 90% O1. The primary emulsion typically comprises at least 1 part W1 to 3 parts O1 and usually at least 1 part W1 to 5 parts O1.

The primary emulsion comprises droplets of the first aqueous phase. The droplets may have a mean diameter of 900 nm or less, 800 nm or less, 700 nm or less, 600 nm or less, 500 nm or less, 400 nm or less, 300 nm or less or 200 nm or less; and/or the droplets may have mean diameter of 10 nm or more, 50 nm or more, 100 nm or more, 200 nm or more, 300 nm or more, 400 nm or more, 500 nm or more, 600 nm or more or 700 nm or more. The primary emulsion may comprise droplets having a mean diameter of from 100 to 600 nm.

In the present specification “mean diameter” refers to a volume mean particle diameter. The volume mean diameter value of the aqueous droplets in the primary emulsion can be measured using the following method:

The emulsion is diluted with a chloroform/hexane mixed solvent (volume ratio: 4/6) to 10 times, then a particle size distribution was measured with a dynamic light scattering nanotrack particle size analyzer (UPA-EX150, manufactured by NIKKISO CO., LTD. or Malvern Zetasizer Nano ZS), and based on this particle size distribution, a volume mean particle diameter were calculated. The volume mean particle diameter of vesicles can be obtained by measuring a suspension of the vesicles in an analogous way using the same device.

The primary emulsion may be prepared by a high shear method (shear rates greater than 10⁵ s⁻¹ usually much higher 10⁷ s⁻¹ or more) or a low shear method (shear rates less than 10⁴ s⁻¹). A low shear method is preferred when encapsulating shear sensitive components.

High shear emulsification methods include the use of a homogenizer or sonication (ultrasonic waves).

Low shear emulsification methods include the use of a nanoporous membrane or nanochannels.

The primary emulsion may be prepared by injecting the first water phase W1 through pores (nanopores) of a hydrophobic nanoporous membrane that is immersed in the oil phase, as illustrated in FIG. 4. The nanoporous membrane may be a glass membrane surface treated with a hydrophobic coating agent or made from another hydrophobic material. Shear at the nanoporous membrane surface allows detachment of the nanometer length scale droplets at the membrane-oil interface; flow of the oil phase across the membrane surface (e.g. by gentle stirring) is sufficient for this purpose.

The size of the pores will determine the size of the droplets of the first aqueous phase in the primary emulsion. Typically the size of the primary emulsion nanodroplets are larger than the membrane pore size e.g. a 100 nm pore will give rise to a 300 nm droplet. The pore size is reported by the manufacturer or can be measured, for example by means of mercury porosimetry. For encapsulation of nanoparticles e.g. bacteriophages, the size of the nanopore needs to be larger than the size of the bacteriophage to avoid filtration of the nanomedicine.

The pore size may be 2000 nm or less, 1500 nm or less, 1000 nm or less, 800 nm or less, 600 nm or less, 500 nm or less, 400 nm or less, 300 nm or less or 200 nm or less; and/or the pore size may be 100 nm or more, 200 nm or more, 300 nm or more, 400 nm or more, 500 nm or more, 600 nm or more, 800 nm or more, 1000 nm or more, or 1500 nm or more. The nanoporous membrane may have a pore size of from 400 to 800 nm.

Typically, the primary emulsion is not prepared by a microfluidic method, whereby the water phase W1 passes through microchannels since the primary emulsion droplets are far too large to yield nanovesicles. The inventors do not consider this to be a feasible method for preparing the primary emulsion for the purposes of producing nanovesicles.

Amphiphilic Membrane Forming Component

The amphiphilic membrane forming component is employed to form the amphiphilic membrane (e.g. bilayer) in the resulting vesicle. The amphiphilic membrane forming component is typically dissolved in the oil phase prior to preparation of the primary emulsion.

Suitable amphiphilic membrane forming components include phospholipids, sphingolipids, glycerolipids, ceramides, sterols, polymerizable lipids, fluorescent lipids, terpenoids, prostaglandins, glycerides, eicosanoids, PAHSA, polysaturated fatty acids, acylcarnitine, alkyl phosphates, natural occurring lipids, cardiolipin, plasmalogens, ether lipids, bacterial lipids, liponucleotides, anandamide, endocannabinoids, acyl CoA, photoswitchable lipids, aliphatic amine, aliphatic acids.

The amphiphilic membrane forming component may be a lipid, such as a phospholipid, so that the resulting vesicle is a liposome having a lipid bilayer. Suitable phospholipids include phosphatidylcholine, phosphatidylserine, phosphatidylglycerol, phosphatidylinositol, phosphatidyethanolamine, and phosphatidic acid.

The amphiphilic membrane forming component may be a polymer, such as a block copolymer, so that the resulting vesicle is a polymerosome.

Block copolymers include diblock copolymers (e.g. PEG diblock copolymer), biodegradable AB diblock copolymers, and polystyrene diblock copolymers.

The amphiphilic membrane forming component may comprise pH-responsive polymers (e.g. PEG-b-PLA), reduction or oxidation-responsive polymer (e.g.PEG-SS-PPS), enzyme-responsive polymer (e.g. (P(GA-co-Ala)-b-PBA), (P(GA-co-Ala)-b-PS)), glucose-responsive (e.g.PEG-b-PBOx), gas-responsive polymer (e.g. PEG-b-PAD), temperature-responsive (e.g.PEG-b-PNIPAM), light-responsive, (e.g.PEO-b-PSPA), triblock copolymer (e.g. biodergadeble ABA triblock copolymers, PEG/PPG triblock copolymers, polystyrene triblock copolymers), amphiphilic block copolymers.

The amphiphilic membrane forming component may comprise PEG-b-PLA, poly(ethylene glycol)-block-poly(D,L-lactic acid).

First Aqueous Phase W1

The first aqueous phase W1 forms the core in the resulting vesicle and can therefore be used to encapsulate a substance, as discussed above.

The first aqueous phase comprises or consists of water.

In addition to water, the first aqueous phase may comprise one or more additives. For example, additives may be employed to adjust the osmotic pressure, density, viscosity or interfacial tension. Suitable additives include inorganic salts (MgSO₄, (NH₄)₂SO₄, NaCl, KCl, etc.), sugar (e.g. sucrose), polymers, surfactants and combinations thereof.

The first aqueous phase may comprise PVA (poly(vinyl) alcohol) and/or PEG (polyethylene glycol), such as PEG-8000.

The first aqueous phase may comprise (w/v) 1% or more, 2% or more or 3% or more PVA; and/or the first aqueous phase may comprise (w/v) 10% or less, 5% or less or 2% or less PVA.

The first aqueous phase may comprise (w/v) 1% or more, 3% or more or 5% or more PEG; and/or the first aqueous phase may comprise (w/v) 20% or less, 12% or less or 8% or less PEG.

The first aqueous phase may comprise a buffer, such as a Tris (tris(hydroxymethyl)aminomethane) buffer, such as a Tris-HCl buffer.

Oil Phase O

The first oil phase O1 forms an oil film located between the first (inner) and second (outer) aqueous phases of the water-in-oil double emulsion. The inventors have determined that there is no need for this oil film to be very thin even for nanovesicles formed through dewetting of double emulsions.

The oil phase (first or subsequent oil phases) comprises or consists of one or more organic solvents. The oil phase may comprise a non-polar organic solvent, a polar aprotic organic solvent and/or a polar protic organic solvent.

Common non-polar organic solvents include pentane, cyclopentane, hexane, cyclohexane, heptane, octane, benzene, toluene, 1,4-dioxane, chloroform, diethyl ether and dichloromethane.

Common polar aprotic organic solvents include tetrahydrofuran (THF), ethyl acetate, acetone, dimethylformamide (DMF), acetonitrile, dimethyl sulfoxide (DMSO), and nitromethane.

Common polar protic organic solvents include formic acid, ethanol, methanol, acetic acid, and propanol.

The oil phase may comprise both a good solvent for the amphiphilic membrane forming component and a poor solvent for the amphiphilic membrane forming component. The difference in solubility is important to allow controlled dewetting and formation of stable nanovesicles.

The oil phase may comprise at least two different organic solvents, a first organic solvent (e.g. chloroform) and a second organic solvent (e.g. hexane). The first and second organic solvents may be selected depending on their properties, including the solubility of the amphiphilic membrane forming component therein, miscibility with water, and density.

The first and/or second organic solvent may be a non-polar solvent.

The first organic solvent may comprise chloroform and/or diethyl ether. Chloroform and diethyl ether allow the amphiphilic membrane forming component to dissolve therein and are water soluble. The water solubility is important since it allows part of the oil phase to diffuse into the aqueous phase, leading to controlled dewetting.

The second organic solvent may comprise an alkane, such as an aliphatic alkane, such as a C5 to C10 (aliphatic) alkane, such as pentane, hexane, heptane and/or octane.

The ratio by volume of the first organic solvent (OS1, e.g. chloroform) to the second organic solvent (OS2, e.g. hexane) may be at least 30% OS1:no more than 70% OS2; at least 40% OS1:no more than 60% OS2; at least 50% OS1:no more than 50% OS2; or at least 60% OS1:no more than 40% OS2.

The oil phase may comprise or consist of chloroform and hexane.

In addition to organic solvent, the oil phase may comprise one or more additives. For example, additives may be employed to adjust the viscosity or interfacial tension.

Suitable additives include polymers, surfactants and chemical compounds.

Second Aqueous Phase W2

The second aqueous phase W2 is employed for dewetting to allow vesicle formation.

The second aqueous phase comprises or consists of water.

In addition to water, the second aqueous phase may comprise one or more additives.

For example, additives may be employed to adjust the osmotic pressure, density, viscosity or interfacial tension. Suitable additives include inorganic salts (MgSO₄, (NH₄)₂SO₄, NaCl, KCl, etc.), sugar (e.g. sucrose), surfactants (including emulsifiers) and combinations thereof.

The second aqueous phase may comprise one or more surfactants, including polymer surfactants. The second aqueous phase may comprise PVA and/or a poloxamer, such as poloxamer 188. Poloxamers are non-ionic triblock copolymers composed of a central hydrophobic chain of polyoxypropylene (poly(propylene oxide)) flanked by two hydrophilic chains of polyoxyethylene (poly(ethylene oxide)).

A poloxomer is not considered suitable for forming vesicles on its own, but may form micelles. A typical micelle in aqueous solution forms an aggregate with hydrophilic “head” regions in contact with the surrounding solvent and hydrophobic tail regions in the micelle centre.

The second aqueous phase may comprise (w/v) 2% or more, 5% or more or 10% or more PVA; and/or the second aqueous phase may comprise (w/v) 20% or less, 15% or less or 10% or less PVA.

The second aqueous phase may comprise (w/v) 1% or more, 3% or more or 5% or more poloxamer; and/or the second aqueous phase may comprise (w/v) 15% or less, 10% or less or 5% or less poloxamer.

Secondary Emulsion

The primary emulsion is combined with a second aqueous phase W2 to produce a secondary emulsion that is a water-in-oil-in-water emulsion. Unilamellar vesicles will form through controlled de-wetting of the oil shell. The kinetics of the process can be controlled through control of the formulation parameters and the resulting vesicles can be separated e.g. by either gravity or centrifugal force, such as using differential centrifugation.

The primary emulsion may be added to an excess of the second aqueous phase. The volume ratio of the primary emulsion (W1/O) to the second aqueous (W2) phase may be 1 to 40% W1/O:99 to 60% W2; 2 to 30% W1/O:98 to 70% W2; 3 to 20% W/O1: 97 to 80% W2; or 5 to 10% W1/O:95 to 90% W2. The volume ratio W1:O may be 1 to 10% W1/O:99 to 90% W2. Typically, the ratio is at least 1 part W1/O to 3 parts W2, usually at least 1 part W1/O to 5 parts W2.

The secondary emulsion may be prepared by pouring the primary emulsion into the second aqueous phase as shown in FIG. 1 or vice versa: the second aqueous phase can be poured into the primary emulsion.

The secondary emulsion may be prepared by injecting the primary emulsion through pores of a porous hydrophilic membrane that is immersed in the second water phase, employing for example a device such as the High-Speed Mini Kit, KH-125; SPG Technology Co., Ltd. The membrane pore size affects the size of the droplets of primary emulsion in the secondary emulsion. It will be understood that a larger pore size will be required for the preparation of the secondary emulsion than for the preparation of the primary emulsion.

The pore size may be 100 μm or less, 50 μm or less, 10 μm or less, 5 μm or less, or 3 μm or less; and/or the pore size may be 0.5 μm or more, 1 μm or more, 3 μm or more, 5 μm or more, or 10 μm or more. The porous membrane for the preparation of the second emulsion may have a pore size of from 4 to 6 μm.

The pore size is reported by the manufacturer or can be measured, for example by means of mercury porosimetry or an electron microscope.

The secondary emulsion may be prepared using a microfluidic channel, an example of which is shown in FIG. 2. The primary emulsion can be passed through an X-junction chip, where the second aqueous phase flows in a direction perpendicular to the primary emulsion. The size of the microfluidic channel determines the size of the droplets in the secondary emulsion.

The microfluidic channel may have a diameter of 500 μm or less, 200 μm or less, 100 μm or less, 50 μm or less, 10 μm or less, 5 μm or less, or 3 μm or less; and/or the microfluidic channel may have a diameter of 1 μm or more, 5 μm or more, 10 μm or more, 50 μm or more or 100 μm or more. The microfluidic channel may have a diameter of from 100 to 300 μm.

The diameter of the microfluidic channel is reported by the manufacturer or can be measured, for example by means of an optical light microscope.

### BRIEF DESCRIPTION OF THE DRAWINGS

FIG. 1: Schematic diagram showing the preparation of a W1/O1/W2 double emulsion by pouring the primary W1/O1 nanoemulsion in an excess of an aqueous phase containing emulsifier to stabilise the vesicle bilayer membrane upon subsequent spontaneous dewetting.

FIG. 2: Schematic diagram showing the preparation of a W1/O1/W2 double emulsion by contacting the primary W1/O1 nanoemulsion with an aqueous phase (W2) in a microcapillary.

FIG. 3: Schematic diagram showing assembly of a unilamellar vesicle from dewetting of the double nanoemulsion.

FIG. 4: Schematic diagram showing the preparation of a W1/O1 nanoemulsion by injecting the inner water phase (W1) through nanopores of a hydrophobic membrane.

FIG. 5 and FIG. 6: Schematic diagrams showing methods to separate the secondary emulsion into layers having different densities.

FIG. 7: Schematic diagram showing a multilamellar vesicle (left) and a multivesicular vesicle (right).

FIG. 8: NTA size analysis of unilamellar liposomes produced using a low shear membrane emulsification process (0.5 μm and 1.1 μm pore glass membranes used to make the W1/O1 primary emulsion).

FIG. 9: CryoTEM image showing unilamellar liposomes (bar 200 nm).

FIG. 10: CryoTEM image showing multivesicular liposomes (bar 200 nm).

FIG. 11: CryoTEM image showing encapsulated phage Mycobacterium D29 in unilamellar liposomes (bar 200 nm).

FIG. 12: CryoTEM image showing unilamellar polymersomes (bar 200 nm).

FIG. 13: CryoTEM image showing multilamellar vesicles (bar 200 nm).

FIG. 14: CryoTEM image showing magnetic nanoparticle encapsulation in liposomes (bar 200 nm).

FIG. 15: CryoTEM image showing a multivesicular liposome (bar 200 nm).

FIG. 16: Confocal images of macrophages exposed to phage K encapsulated in DOPE-CHEMS liposomes. After growth, macrophages were exposed to phage K encapsulated in DOPE-CHEMS liposomes, then permealised and stained. Phage K was pre-stained with SYBR gold (left, green in original image). Nuclei are stained with DAPI (left centre, blue in original image). Actin filaments are stained with phalloidin/CFR680 Cy5 fluorophore conjugate (right centre, red in original image). Image on the right is the merged image. Error bar 10 μm.

FIG. 17: Confocal imaging of macrophages exposed to S. aureus and then treated with CHAP_{K} and LLO encapsulated in DOPE-CHEMS liposomes. After growth, macrophages were exposed to S. aureus and then treated with DOPE-CHEMS liposomes loaded with CHAP_{K} and LLO, then permealised and stained. Nuclei are stained with DAPI (left, blue in original image). Actin filaments are stained with phalloidin/CFR680 Cy5 fluorophore conjugate (centre, red in original image). Image on the right is the merged image. Top row=control, bottom row=treated sample. Error bar 10 μm.

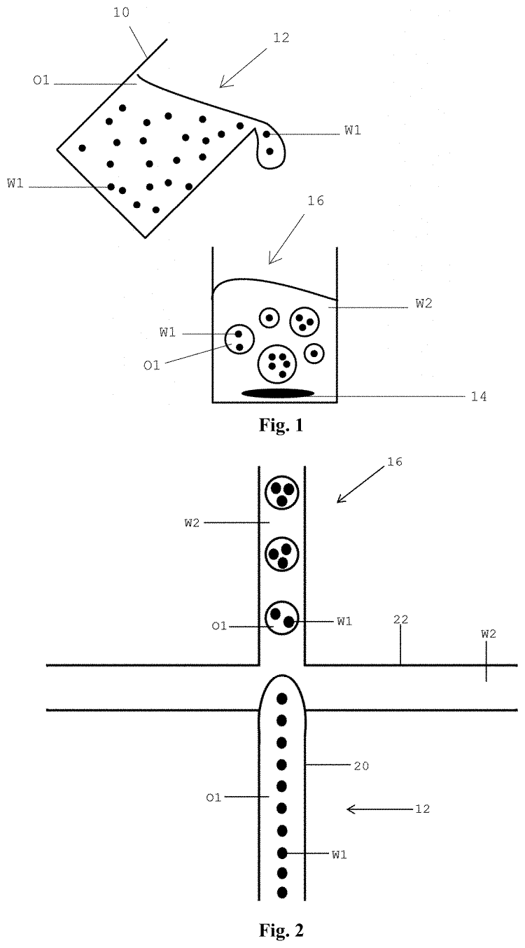

Referring to FIG. 1, there is shown a schematic diagram of a process in accordance with an embodiment of the invention. A container 10 holds a primary emulsion 12 therein. The primary emulsion 12 comprises droplets of a first aqueous phase W1 dispersed in a continuous oil phase O1. The primary emulsion 12 is poured into an excess of a second aqueous phase W2 with stirring provided by a magnetic bar 14. A secondary emulsion 16 is formed, which is a water-in-oil-water emulsion. The secondary emulsion 16 comprises droplets of the first emulsion 12 dispersed in the second aqueous phase W2. It is notable that the droplets of the primary emulsion contain one or more droplets of the first aqueous phase W1.

Referring to FIG. 2, there is shown a schematic diagram of another process to prepare the secondary emulsion 16. As before, a primary emulsion 12 comprises droplets of the first aqueous phase W1 dispersed in a continuous oil phase O1. The primary emulsion is pumped through a microcapillary channel 20 and the second aqueous phase W2 flows perpendicular to the primary emulsion, through another channel 22, thereby forming an X junction. The primary emulsion 12 and the second aqueous phase W2 combine to form the secondary emulsion 16.

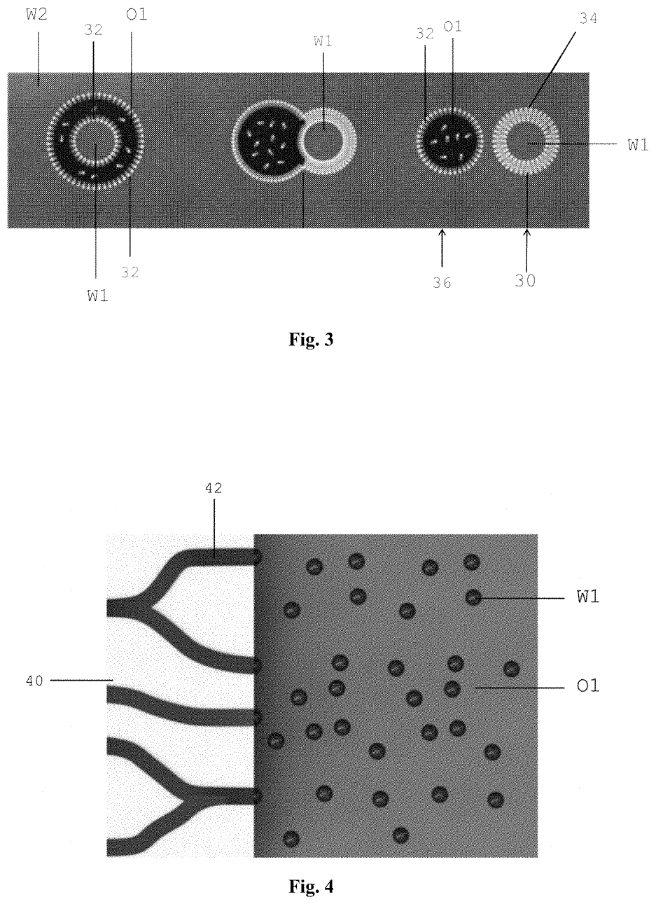

Referring to FIG. 3, there is shown a schematic diagram showing assembly of a unilamellar vesicle 30 from dewetting of a double nanoemulsion.

On the left there is shown a droplet of a first aqueous phase W1, which is enclosed within a droplet of a first oil phase O1, which is dispersed in a continuous second aqueous phase W2. An amphiphilic membrane forming component 32 is present, which tends to aggregate at the interface between the oil and water phases.

In the middle, there is shown partial dewetting. Without being bound by theory, a portion of the oil phase, e.g. a water-soluble organic solvent, is believed to migrate out of the oil phase and this depletion leads the remaining oil phase components to separate, perhaps through a difference in density or due to Brownian thermal motion.

On the right there is shown a unilamellar liposome 30 comprising an amphiphilic membrane 34 enclosing an aqueous core W1. There is also shown an oil droplet 36 comprising oil phase O1 and excess amphiphilic membrane forming component 32.

### EXAMPLES

### Example 1: Generation of W1/O1 Nanoemulsion Using a Low Shear Primary Emulsification Process, for the Subsequent Preparation of Liposomes

1.5 g of phosphatidylcholine (amphiphilic membrane forming component) was dissolved in 20 ml of a solvent mixture (chloroform/hexane volume ratio 2:3) and used as the organic phase (O1). PVA (2% w/v), PEG-8000 (6% w/v) and a Myovirus Staphylococcus aureus phage K were solubilized in Tris-HCl buffer (W1) to form the first aqueous phase.

Production of the W1/O1 nanoemulsion was carried out using a nanoporous glass membrane 40 (i.e. membrane emulsification) as illustrated in FIG. 4. The water phase was passed through the porous membrane to form nanodroplets at the membrane surface where they then detach under low shear and were dispersed in the oil phase.

The nanoporous glass membrane (SPG Technology Co., Ltd, Japan) 40 comprises pores 42 having a pore size of 500 nm. The nanoporous membrane 40 was immersed in the oil phase (O1) and kept under constant gentle stirring to provide gentle shear across the membrane surface. 5 ml of the inner aqueous phase (W1) was transferred to 20 ml of the oil phase (O1) by pushing the water phase W1 under pressure (1 to 10 times greater than the critical pressure) across a 2 cm length of the tubular hydrophobic nanoporous glass membrane. This W1/O1 nanoemulsion was kept stirring throughout.

### Example 2: Generation of W1/O1 Nanoemulsion Using a Low Shear Primary Emulsification Process, for the Subsequent Preparation of Polymerosomes

Example 1 was repeated with 0.5 g PEG(5000)-b-PLA(5000) in place of the 1.5 g of phosphatidylcholine as the amphiphilic membrane forming component. 1 ml of the inner aqueous phase (W1) transferred to 20 ml of the oil phase (O).

### Example 3: Generation of W1/O1 Nanoemulsion by Primary Emulsification Step

1.5 g of phosphatidylcholine was dissolved in 20 ml of a solvent mixture (chloroform/hexane 2/3) and used as the organic phase (O1). 5 ml of the first aqueous phase (W1; PVA 2% w/v, PEG-8000 6% w/v and calcein in Tris-HCl buffer) was added to the oil phase in a beaker with a paddle mixer to form a premix microemulsion of W1 droplets dispersed in the oil phase.

The premix was subsequently emulsified at 1200 psi using a single pass homogenization cycle (Microfluidizer LV1) yielding a primary emulsion W1/O1 with droplets between 50 nm-500 nm in size measured using dynamic light scattering (Malvern Zetasizer Nano ZS).

### Example 4: Generation of W1/O1 Nanoemulsion by Primary Emulsification Step

1.5 g of phosphatidylcholine was dissolved in 20 ml of a solvent mixture (chloroform/hexane 2/3) and used as the organic phase (O). 5 ml of the first aqueous phase (W1; PVA 2% w/v, PEG-8000 6% w/v and calcein in Tris-HCl buffer) were added to the oil phase in a beaker. Production of the W1/O1 primary emulsion was carried out using an ultrasonic probe for emulsification (Hielscher UP200St) yielding a nanoemulsion W1/O1 with droplets between 50 nm-500 nm in size (measured using dynamic light scattering (Malvern Zetasizer Nano ZS).

### Example 5: Generation of W1/O/W2 Double Emulsion and Separation

As illustrated in FIG. 1, 25 ml of the primary W1/O1 emulsion prepared using one of the methods described above (examples 1-4) was poured into 75 ml of the second aqueous phase (the outer phase W2; PVA 10% w/v, Poloxamer-188 5% w/v) under gentle stirring to form the secondary emulsion, which is a water-in-oil-in-water emulsion, i.e. a double emulsion. De-wetting of the double emulsion occurs over a period of several minutes at room temperature. Poloxamer-188 is used to adjust the interfacial tension between phases and thereby aid dewetting.

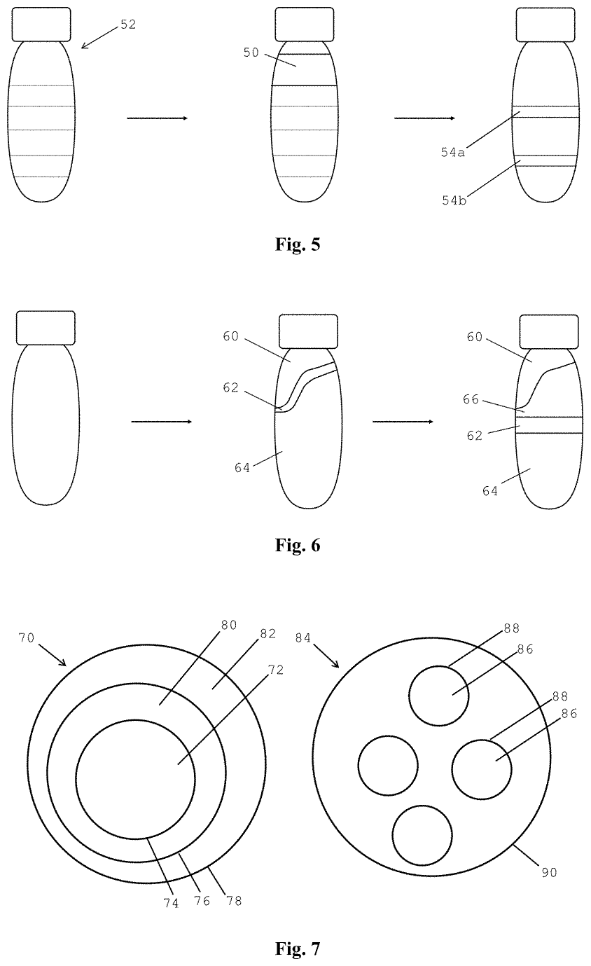

Referring to FIG. 5, 10 ml of the secondary emulsion 50 now containing the nanovesicles was aliquoted in a 50 ml centrifuge tube and layered on a 40 ml sucrose gradient 52 (layers 10 wt %, 20 wt %, 30 wt % and 40 wt %) and centrifuged at 50,000 g (Beckman Coulter, Model Avanti JXN-30) for 12 hours at 4° C. Two vesicle rich bands 54a, 54b were generated. The “empty” nanovesicles, which do not encapsulate a nanoparticle are less dense and are present in the upper layer 54a. The nanovesicles with cargo are denser and present in the lower layer 54b. Each vesicle rich layer 54a, 54b was removed using a syringe to pierce the tube and dialysed several times using a 12-14 kDa dialysis membrane and resuspended in Tris-HCl buffer. Vesicles were stored at 4° C.

Alternative gradients, such as a CsCl (caesium chloride) gradient, can be used in place of a sucrose gradient.

Another option is illustrated in FIG. 6 wherein the double emulsion comprising the vesicles is first centrifuged to form three layers of different densities: an oil layer 60, a vesicle rich layer 62 and an aqueous layer 64. A sucrose solution 66 (e.g. 100 mM sucrose) is then added to separate the oil layer 60 from the vesicle rich layer 62. The diagram is provided for illustration only and is not to scale.

### Example 6: Generation of W1/O1/W2 Double Emulsion Using Membrane Emulsification, and Subsequent Separation

The primary W1/O1 emulsion prepared using one of the methods illustrated above (examples 1-4) was passed through a porous membrane (e.g. using a High-Speed Mini Kit, KH-125; SPG Technology Co., Ltd.) mounted with a 5 μm pore size hydrophilic membrane and dispersed in the outer phase (W2; PVA 10% w/v, Poloxamer-188 5% w/v). De-wetting of the double emulsion occurs over a period of several minutes at room temperature and vesicles were isolated as described above.

### Example 7: Generation of Vesicles by Preparing a W1/O1/W2 Double Emulsion in a Microfluidic Channel

As illustrated in FIG. 2, the primary W1/O1 emulsion prepared using one of the methods illustrated above (examples 1-4) was pumped (Vici pump, equipped with silicone tubing) into a quartz droplet X-junction chip (190 m, hydrophilic) and emulsified in a second aqueous phase (outer aqueous phase W2; PVA 10% w/v, Poloxamer-188 5% w/v). De-wetting of the double emulsion occurs over a period of several minutes at room temperature and vesicles were separated as described above.

### Example 8: Generation of Bilamellar and Multilamellar Liposomes

1.5 g of phosphatidylserine was dissolved in 20 ml of a solvent mixture (chloroform/hexane volume ratio 2:3) and used as a second organic phase (O2). PVA (2% w/v), PEG-8000 (6% w/v) and unilamellar liposomes produced using one of the methods illustrated above were suspended in Tris-HCl buffer (W3). Production of a W3/O2 nanoemulsion was carried out using a 1 μm hydrophobic porous glass SPG membrane. The W3/O2 nanoemulsion was poured into the outer aqueous phase (W4; PVA 10% w/v, Poloxamer-188 5% w/v) under gentle stirring. De-wetting of the double emulsion occurs over a period of several minutes at room temperature. 10 ml of the secondary emulsion (nanovesicle mixture) was aliquoted in a 50 ml centrifuge tube and layered on a 40 ml sucrose gradient (layers 10 wt %, 20 wt %, 30 wt % and 40 wt %) and centrifuged at 50,000 g (Beckman Coulter, Model Avanti JXN-30) for 12 hours at 4° C. The vesicle rich band was removed using a syringe to pierce the tube and dialysed several times using a 12-14 kDa dialysis membrane and resuspended in Tris-HCl buffer. Vesicles were stored at 4° C. Multilamellar vesicles were stored at 4° C.

FIG. 7 shows schematic diagrams of a multilamellar vesicle 70 (left) and a multivesicular vesicle 84 (right).

The multilamellar vesicle 70 comprises a central aqueous core 72 surrounded by an inner amphiphilic membrane 74, an intermediate amphiphilic membrane 76 and an outer amphiphilic membrane 78. The region between the inner and intermediate amphiphilic membranes can be described as a first shell 80 and the region between the intermediate and outer amphiphilic membranes can be described as a second shell 82.

The multivesicular vesicle 84 comprises four aqueous cores 86 with each core being enclosed by an inner amphiphilic membrane 88. Each core 86 and inner amphiphilic membrane 88 can be viewed as a unilamellar vesicle, which is enclosed within an outer amphiphilic membrane 90.

NTA Size Analysis

A NanoSight LM10 (Malvern Instruments Ltd, UK) using nanoparticle tracking analysis (NTA) was used to determine the average size and size distribution of liposomes/polymerosomes. NTA measurements were performed in a sample chamber equipped with a 640 nm LASER to track the nanoparticles (NPs).

Typically, a 10 μl aliquot was taken from each sample and diluted 10²-10³ fold in order to achieve a particle concentration of 10⁷-10¹⁰ ml⁻¹. The sample was injected into the sample chamber using a sterile syringe and sample flow was maintained through the chamber until all air bubbles were removed. The temperature was registered with a thermometer (RTD Pt100, OMEGA, UK) and temperature correction was carried out.

The software used for capturing and analysing the data was NTA 3.0 (Malvern Instruments Ltd, UK). Data for each sample was captured over a period of 60 s and each measurement was repeated five times.

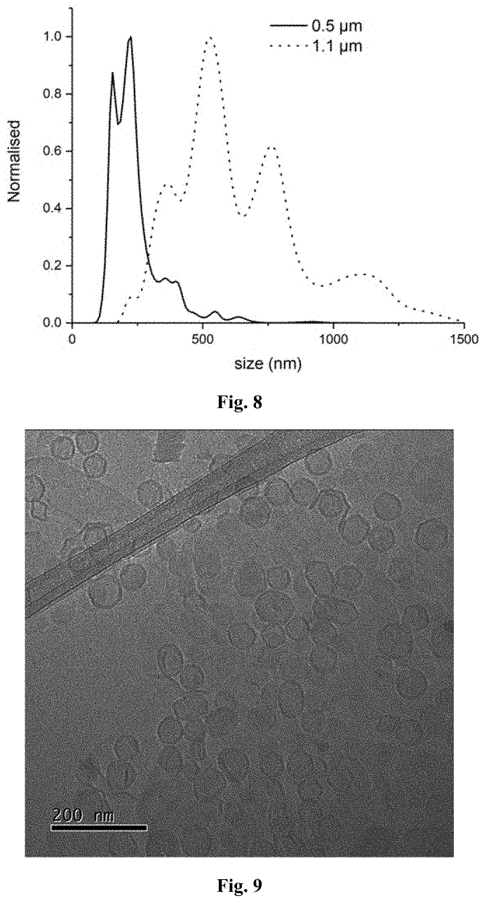

The focus was set to achieve a uniform perfect spherical particle view. Before capturing the video, the camera had to be set-up to ensure all the particles in the sample were clearly visible with no more than 20% saturation. The single gain mode was used throughout the whole measurement process. An example for unilamellar liposomes is shown in FIG. 8.

CryoTEM Imaging

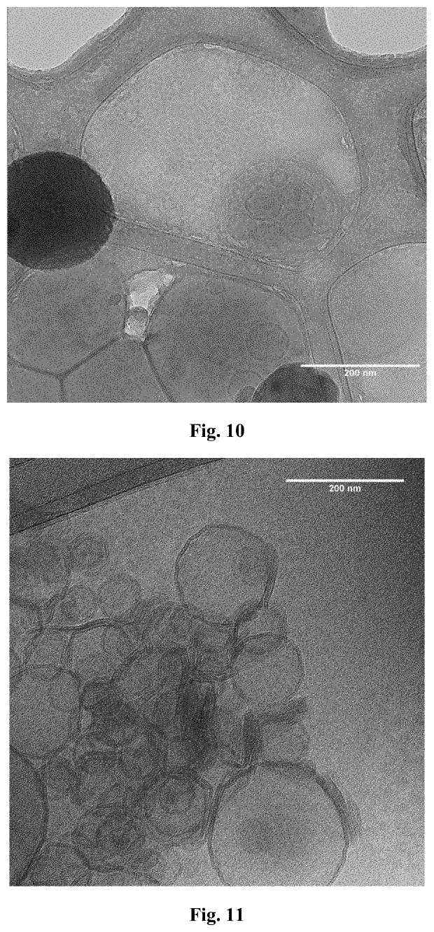

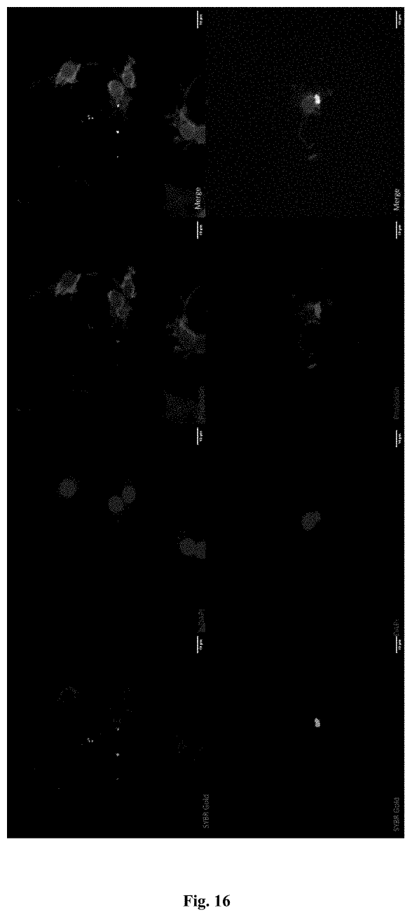

A 8 μl aliquot of sample was pipetted onto a carbon coated copper grid (HC300Cu, Holey Carbon film on Copper 300 mesh, EM Resolutions, UK). Excess liquid was blotted away with filter paper (Whatman number 1) and the grid was plunge-frozen in a liquid mixture of ethane/propane cooled by liquid nitrogen. The sample was then kept at liquid nitrogen temperatures throughout the analysis. TEM images were taken on a JEOL 2200FS TEM at 200 keV using a Gatan K2 Summit and Gatan 914 cryo-holder. A selection of images is shown in FIGS. 9 to 15.

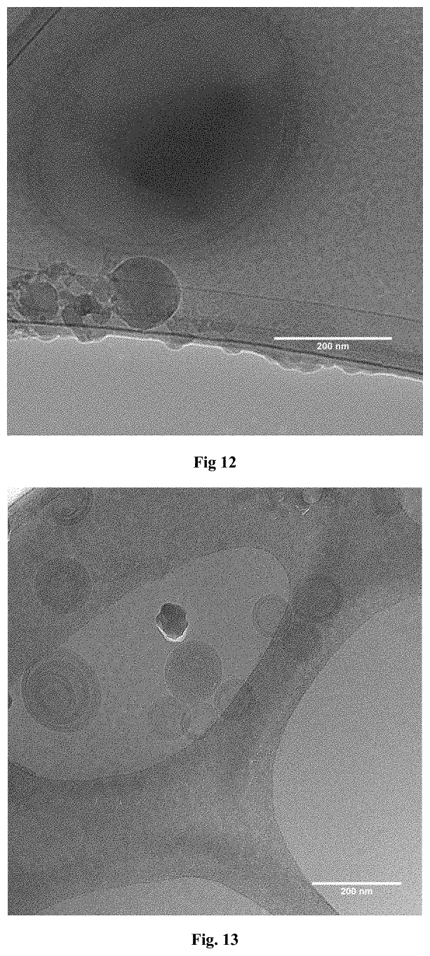

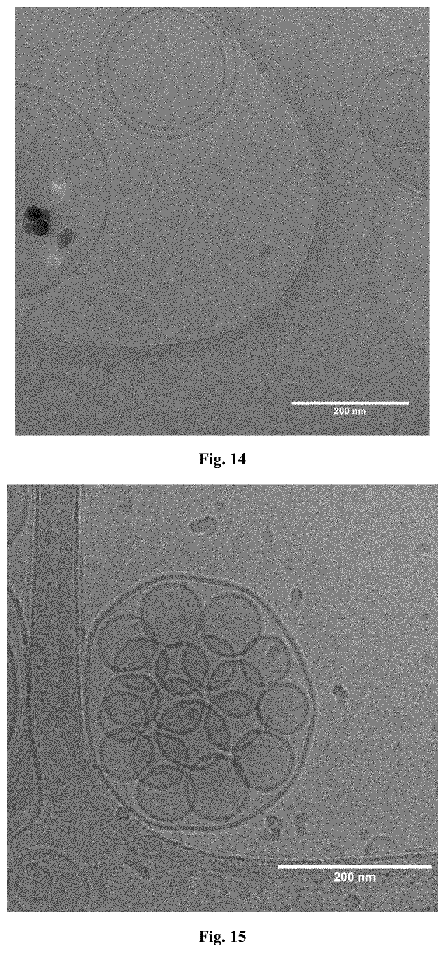

FIG. 11 demonstrates encapsulation of a phage D29. FIG. 13 clearly shows the concentric ring pattern of a multilamellar vesicle. Referring to FIG. 14, there is shown a CryoTEM image showing encapsulation of a magnetic nanoparticle (MNP, iron oxide) in liposomes. MNPs are used in oncology for drug delivery and as contrast agents for magnetic resonance.

### Example 9

DOPE-CHEMS liposomes are composed of dioleoylphosphatidylethanolamine (DOPE) and cholesteryl hemisuccinate (CHEMS).

We encapsulated CsCl purified S. aureus myoviridae phage K in DOPE-CHEMS liposomes using the method described in example 5 and imaged phage and macrophage compartments by fluorescent staining and confocal microscopy.

Briefly, 1.5 g of lipids DOPE-CHEMS (2:1 molar ratio) was dissolved in 20 ml of a solvent mixture (chloroform/hexane volume ratio 2:3) and used as the organic phase (O1). PVA (2% w/v), PEG-8000 (6% w/v) and a Myovirus Staphylococcus aureus phage K were solubilized in Tris-HCl buffer (W1) to form the first aqueous phase.

Production of the W1/O1 nanoemulsion was carried out using a nanoporous glass membrane 40 (i.e. membrane emulsification) as illustrated in FIG. 4. The water phase was passed through the porous membrane to form nanodroplets at the membrane surface where they then detach under low shear and were dispersed in the oil phase.

The nanoporous glass membrane (SPG Technology Co., Ltd, Japan) comprises pores having a pore size of 1.1 μm. The nanoporous membrane was immersed in the oil phase (O1) and kept under constant gentle stirring to provide gentle shear across the membrane surface. 5 ml of the inner aqueous phase (W1) was transferred to 20 ml of the oil phase (O1) by pushing the water phase W1 under pressure (1 to 10 times greater than the critical pressure) across a 2 cm length of the tubular hydrophobic nanoporous glass membrane. This W1/O1 nanoemulsion was kept stirring throughout.

As illustrated in FIG. 1, 25 ml of the primary W1/O1 emulsion was poured into 75 ml of the second aqueous phase (the outer phase W2; PVA 10% w/v, Poloxamer-188 5% w/v) under gentle stirring to form the secondary emulsion. De-wetting of the double emulsion occurs over a period of several minutes at room temperature. Poloxamer-188 was used to adjust the interfacial tension between phases and thereby aid dewetting.

Analogous to FIG. 5, 10 ml of the secondary emulsion now containing the nanovesicles was aliquoted in a 50 ml centrifuge tube and layered on a 40 ml sucrose gradient (layers 10 wt %, 20 wt %, 30 wt % and 40 wt %) and centrifuged at 50,000 g (Beckman Coulter, Model Avanti JXN-30) for 12 hours at 4° C. Two vesicle rich bands were generated. The “empty” nanovesicles, which do not encapsulate phage K are less dense and are present in the upper layer. The nanovesicles with cargo are denser and present in the lower layer 54b. Each vesicle rich layer was removed using a syringe to pierce the tube and dialysed several times using a 12-14 kDa dialysis membrane and resuspended in Tris-HCl buffer. Vesicles were stored at 4° C.

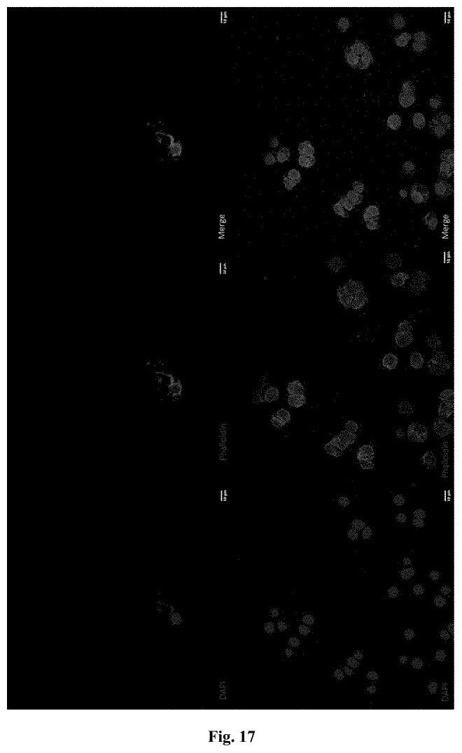

DOPE-CHEMS liposomes were internalized to a high level as we imaged a high intracellular concentration of fluorescent phage inside the macrophages. Upon uptake of phage K in DOPE-CHEMS liposomes, macrophage cells (FIG. 16) were still viable; indicating that encapsulated phage K was not cytotoxic to the macrophages.

FIG. 16 shows intracellular delivery of a large myophage (phage K) encapsulated in liposomes into a human macrophage cell line.

### Example 10

We co-encapsulated LLO and S. aureus specific endolysin CHAP_{K} in pH-responsive DOPE-CHEMS liposomes using the method described in example 9. Briefly, 1.5 g of lipids DOPE-CHEMS (2:1 molar ratio) was dissolved in 20 ml of a solvent mixture (chloroform/hexane volume ratio 2:3) and used as the organic phase (O1). PVA (2% w/v), PEG-8000 (6% w/v) containing LLO (200 μg ml⁻¹) and purified CHAP_{K} (50 μg ml⁻¹) in SM buffer (W1) to form the first aqueous phase. Production of the W1/O1 nanoemulsion was carried out using a 500 nm nanoporous glass membrane (i.e. membrane emulsification) as per the procedure outlined in example 9.

Macrophages were infected with a deadly concentration of S. aureus and then treated with either empty liposomes as a control (FIG. 17, top row) or LLO/CHAP_{K} liposomes (FIG. 17, bottom row).

In the former sample, we found that only few living cells (FIG. 17, top row) and those that are alive are heavily infected with S. aureus (as shown by cell morphology and presence of intracellular S. aureus). In the endolysin-treated sample, instead, we found a high number of cells with normal morphology, implying recovery from the infection, and decreased occurrence of intracellular S. aureus.

FIG. 17 shows recovery of macrophages treated with a lethal dose of Staphylococcus aureus and then delivery of encapsulated endolysin and its release from the macrophages using a co-encapsulated haemolysin LLO which breaks the liposome upon acidification of the endosome.

### REFERENCES

Deng, N. N., Yelleswarapu, M., and Huck, W. T. S. (2016). Monodisperse Uni- and Multicompartment Liposomes. J. Am. Chem. Soc. 138, 7584-7591.
Ding, S., Anton, N., Akram, S., Er-Rafik, M., Anton, H., Klymchenko, A., et al. (2017). A new method for the formulation of double nanoemulsions. Soft Matter 13, 1660-1669.
Huang, Y., Kim, S. H., and Arriaga, L. R. (2017). Emulsion templated vesicles with symmetric or asymmetric membranes. Adv. Colloid Interface Sci. 247, 413-425.
Rasmussen M K, Pedersen J N, Marie R. Size and surface charge characterization of nanoparticles with a salt gradient. Nat Commun. 2020; 11(1):2337. Published 2020 May 11.
Szoka, F., and Papahadjopoulos, D. (1978). Procedure for preparation of liposomes with large internal aqueous space and high capture by reverse-phase evaporation. Proc. Natl. Acad. Sci. U.S.A 75, 4194-4198.

1. A process for the preparation of unilamellar vesicles, a unilamellar vesicle comprising an amphiphilic membrane enclosing an aqueous core, the process comprising
providing a primary emulsion comprising an amphiphilic membrane forming component, a first aqueous phase W1 and a first oil phase O1, the first aqueous phase W1 being dispersed as droplets in the oil phase O1 such that the primary emulsion is a water-in-oil emulsion and wherein the primary emulsion comprises droplets having a mean diameter of less than 1000 nm;
combining the primary emulsion with a second aqueous phase W2 to produce a secondary emulsion that is a water-in-oil-in-water emulsion;
dewetting to yield unilamellar vesicles in the secondary emulsion; and
isolating the unilamellar vesicles.

2. The process of claim 1, wherein isolating the unilamellar vesicles comprises separating the secondary emulsion into layers of different densities, at least one layer comprising the first oil phase; at least one layer comprising the second aqueous phase; and at least one layer comprising the unilamellar vesicles.

3. The process of claim 1, wherein isolating the unilamellar vesicles comprises (i) filtration; (ii) chromatography; (iii) the use of a salt gradient; and/or (iv) field flow fractionation.

4. The process off claim 1, wherein the first aqueous phase comprises a nanoparticle, such that at least one unilamellar vesicle encapsulates a nanoparticle.

5. The process of claim 4, wherein the nanoparticle is selected from a bacteriophage, a plasmid, an endolysin or a gene vector.

6. The process of claim 1, wherein providing the primary emulsion comprises preparing the primary emulsion by means of a low shear emulsification process.

7. The process of claim 6, wherein the low shear emulsification process employs a nanoporous membrane or nanochannels.

8. The process of claim 1, wherein dewetting to yield unilamellar vesicles in the secondary emulsion does not comprise evaporation of the oil phase.

9. The process of claim 2, wherein a portion of the secondary emulsion comprising the unilamellar vesicles is transferred to a centrifuge tube.

10. The process of claim 2, wherein a density gradient is employed to separate the secondary emulsion into layers of different densities.

11. The process of claim 2, wherein separating the secondary emulsion into layers of different densities comprises centrifuging the secondary emulsion.

12. The process of claim 1, wherein the second aqueous phase comprises PVA (poly(vinyl) alcohol) or a poloxamer.

13. The process of claim 1, wherein the amphiphilic membrane forming component comprises (i) a lipid, such as a phospholipid; or (ii) a di-block copolymer, such as a PEG di-block copolymer.

14. The process of claim 1, wherein the first oil phase comprises a first organic solvent and a second organic solvent.

15. (canceled)

16. (canceled)

17. The process of claim 1, wherein the unilamellar vesicles are further processed to generate multilamellar vesicles.

18. The process of claim 17, wherein said further processing comprises emulsifying a third aqueous phase comprising the unilamellar vesicles with a second oil phase to form a water-in-oil emulsion and subsequently emulsifying with a fourth aqueous phase to form a water-in-oil-in-water emulsion.

19. A composition comprising layers of different densities;
at least one layer comprising an oil phase (O1);
at least one layer comprising an aqueous phase (W2); and
at least one layer comprising a plurality of unilamellar vesicles, the unilamellar vesicles each comprising an amphiphilic membrane enclosing an aqueous core and having a mean diameter of less than 1000 nm.

20. The composition of claim 19, comprising two or more layers comprising a plurality of unilamellar vesicles.

21. The composition of claim 19, wherein at least one unilamellar vesicle has a diameter of less than 1000 nm and encapsulates a nanoparticle having a mean diameter of 10 to 200 nm.

22. (canceled)

23. (canceled)

24. A multilamellar vesicle comprising two or more amphiphilic membranes successively enclosing an aqueous core, the multilamellar vesicle having a diameter of less than 1000 nm and encapsulating a nanoparticle having a mean diameter of 10 to 200 nm.

25. (canceled)
